(19) United States
(12) Patent Application Publication
(10) Pub. No.: US 2024/0240118 A1
(43) Pub. Date: Jul. 18, 2024

(54) SYSTEMS AND METHODS FOR PRODUCING ADDITIONAL DISTILLERS CORN OIL AND A HIGH PROTEIN PRODUCT FROM AN ETHANOL PRODUCTION FACILITY

(71) Applicant: Cellulosic Ethanol Technologies, LLC, Galva, IA (US)

(72) Inventor: Ye-Mon Chen, Galva, IA (US)

(21) Appl. No.: 18/411,546

(22) Filed: Jan. 12, 2024

Related U.S. Application Data
(60) Provisional application No. 63/480,116, filed on Jan. 17, 2023.
(60) Provisional application No. 63/611,266, filed on Dec. 18, 2023.

(51) Int. Cl. C12F 3/10; A23D 9/02; A23K 10/38; C11B 1/10; C12P 7/06

(52) U.S. Cl. CPC C12F 3/10; A23D 9/02; A23K 10/38; C11B 1/10; C12P 7/06

(57) ABSTRACT

A method of producing additional distillers corn oil and a high protein product from an ethanol production facility includes obtaining a supply of whole stillage from the ethanol production facility after ethanol has been extracted therefrom; and introducing at least one diluent into the whole stillage, wherein the diluent has a lower solid percentage than the whole stillage, and the mixture of the whole stillage and at least one diluent produces a distillers corn oil containing diluted stillage product.

### RELATED APPLICATIONS

The present non-provisional patent application claims priority benefit with regard to all common subject matter of prior-filed U.S. provisional patent applications: Ser. No. 63/480,116, filed Jan. 17, 2023, SYSTEMS AND METHODS FOR PRODUCING ADDITIONAL DISTILLERS CORN OIL AND HIGH PROTEIN CORN MEALS FROM AN ETHANOL PRODUCTION FACILITY'S WHOLE STILLAGE; and Ser. No. 63/611,266, filed Dec. 18, 2023, SYSTEMS AND METHODS FOR PRODUCING ADDITIONAL DISTILLERS CORN OIL AND HIGH PROTEIN CORN MEALS FROM AN ETHANOL PRODUCTION FACILITY'S WHOLE STILLAGE. The above-referenced prior-filed patent applications are hereby incorporated by reference into the present patent application in their entireties.

### BACKGROUND

The production of ethanol for use as a gasoline additive or a straight liquid fuel continues to increase as petroleum costs rise and environmental concerns about fossil fuel's high carbon emissions become more pronounced. Conventional ethanol may be produced in a dry milling process by grinding corn or other grains into a powder or flour and then liquefying, fermenting, and distilling it to produce alcohol, carbon dioxide, and conventional whole stillage, a complex multi-phase mixture with high viscosity, which includes water, fibers, distiller corn oil, protein and others. Conventional ethanol production processes produce ethanol from the starch portion of the grain but do not produce additional ethanol from the fiber portions of the grain, which are primarily found in the byproducts. Instead, the byproducts are typically dehydrated and used as low value animal feed, called Distiller's Dried Grain with Soluble, or DDGS.

U.S. Pat. No. 8,633,003, the teachings of which are incorporated by reference into the present application in their entirety, discloses systems and methods for producing additional cellulosic ethanol from the fiber potions of grains in whole stillage, which, as mentioned above, are mostly unused byproducts of existing ethanol production facilities. The technology disclosed in of U.S. Pat. No. 8,633,003 produces more distillers corn oil (DCO) than conventional ethanol plants, via converting a portion of the fibers in the conventional whole stillage. DCO produced from ethanol plants has become a premium feedstock for producing renewable diesel and low carbon sustainable aviation fuel (SAF).

Publication #73285 of USDA Agricultural Research Service (May 1, 1997) describes the use of a process to separate the corn germ from the rest of the corn that has been ground and sieved to a size suitable for protein extraction. The milled corn is then mixed with an aqueous solution of specific gravity equal to the germ and pumped to a hydrocyclone. In the hydrocyclone, the particles with low inertia, that is, the lighter particles which includes the germ-rich particles, leave suspended in the overflow. The rest of the corn particles are driven by centrifugal force to the hydrocyclone underflow.

U.S. Pat. No. 8,778,433, discloses methods for producing a high protein corn meal from a whole stillage byproduct. The methods include complex, multiple stages of separating the whole stillage byproducts, first into an insoluble solids portion and a thin stillage portion. Then, the thin stillage portion is separated into a protein portion and a water-soluble solids portion. The protein portion is dewatered then dried to a high protein corn meal.

U.S. Pat. No. 9,516,891 discloses an even more complex multi-sage process, including the use of special chemicals, for separation of biocomponents from whole stillage into oil, protein, fiber and water. In the first step, fibers are separated in a process that includes a plate separation process and a press. In a subsequent step, the liquid stream separated from the fibers, which contains oil, protein and water, is treated with a special chemical composition that causes the protein to gel. The liquid stream is then processed in a phase separator that drains the oil by gravity, removes the water by an impeller under pressure, and removes the solidified protein using a scroll.

To produce DCO, the majority of conventional ethanol plants, including ethanol plants with the prior art of U.S. Pat. No. 8,633,003, start the backend DCO production process with a two-phase centrifuge solid separation device to produce a solid phase of wet distillers grain (WDG), or commonly known as the wet cake, and a liquid phase of thin stillage, recycling a portion of thin stillage as backset to the front-end of the corn ethanol plant, followed by producing DCO from the remaining portion of thin stillage, or syrup, via evaporation of thin stillage to concentrate DCO in syrup. The shortcoming of this approach is that during the first step of this backend DCO production process with a two-phase centrifuge solid separation device, such as a decanter, a significant portion of DCO in the whole stillage is “lost forever” to the solid phase of wet cake, which becomes no longer available for the DCO production from the liquid phase of thin stillage or syrup. None of the prior arts referenced above address this cause of high DCO loss to the wet cake, leading to low DCO yield from the current corn ethanol plants, except for an ethanol facility constructed in accordance with U.S. Pat. No. 8,633,003, also known as KFT, which had limited success.

Table 1 below shows the Q3.2020 benchmark data of 46 US ethanol plants with combined capacity of 4.3 billion gallons per year. The medium DCO yield is 0.79 lb/bushel, which is slightly higher than previous benchmarks. Corn from different US regions has different DCO content; on average, there is about 2 lb/bushel of DCO in corn. This means that the current ethanol industry is producing only ˜ 40% of DCO as a premium product from corn and the remaining ˜ 60% DCO in corn ends up in the low protein and low value product of DDGS.

| | Year-Quarter | | |
|---|---|---|---|
| Metric | 2020-Q1 | 2020-Q3 | 2021-Q1 |
| Number of Plants | 57 | 48 | 46 |
| Denatured Gal/Bu Ethanol Yield | 2.97 | 2.95 | 2.95 |
| Gallons of Production Capacity | 5638 | 4604 | 4337 |
| Median Plant Size | 100 | 99 | 75 |
| Median Corn Oil Yield (lb/bu) | 0.72 | 0.74 | 0.79 |
| Median Total Enz & Yeast (c/Dgal) | 3.89 | 3.73 | 3.64 |
| Median HPLC (% wt/vol) Ethanol | 14.39 | 14.24 | 14.41 |
| Median HPLC (% wt/vol) Glycerol | 1.00 | 1.04 | 0.99 |

### SUMMARY

The present invention solves the above-described problems and related problems and provides distinct advantages over the current state-of-art of DCO production processes for ethanol production facilities. More specifically, the present invention provides processes and systems for producing additional DCO and a high protein product with conventional ethanol production facilities and ethanol production facilities constructed in accordance with prior art U.S. Pat. No. 8,633,003.

The current invention follows a unique and effective strategy of reducing the DCO loss to the wet cake via processes and methods of reducing the amount of DCO being sent into a two-phase centrifuge solid separation device, such as a decanter. Instead of starting with a solid centrifuge separation device to separate the whole stillage into wet cake and thin stillage as the first step in DCO production, the present invention provides unique processes and methods for reducing the amount of DCO being sent into the decanter.

An objective of the current invention is to significantly increase DCO yield from a corn ethanol plant by following a unique and effective strategy of reducing DCO loss to the wet cake. The present invention achieves these objectives and others by inserting at least one diluent and additional shearing flow separator systems in between the whole stillage and the first solid separation device, such as a decanter, for making the wet distillers grain, or wet cake, and hence is able to significantly reduce the amount of DCO being sent into the decanter, which leads to significant increase in overall DCO production.

Another objective of the current invention is to produce a high protein product in the same processes and systems for significantly increasing DCO yield. The present invention achieves these objectives and others by inserting additional separator systems in between the whole stillage and the first solid centrifuge separation device. These additional shearing flow separator systems isolate fine particles as a product, which have high protein, from the rest of other solids.

An embodiment of the invention is a method of producing additional distillers corn oil from an ethanol production facility. The method comprises obtaining a supply of whole stillage from an ethanol production facility after ethanol has been extracted therefrom: and introducing at least one diluent into the whole stillage, wherein the diluent has a lower percentage of solid than the whole stillage. The mixture of the whole stillage and the diluent produces a distillers corn oil containing diluted stillage product the method further comprises introducing the first distillers corn oil containing diluted stillage product to a first shearing flow separator, wherein the first shearing flow separator produces a first overflow stillage product and a first underflow stillage product, wherein the first overflow stillage product has a higher percentage of distillers corn oil and a lower percentage of solid than the first underflow stillage product. The method further comprises introducing the first underflow product to a solid centrifuge separator, wherein the solid separator produces a thin stillage and a wet distiller grain, or wet cake, with reduced distillers corn oil loss.

Another embodiment of the invention is a process for producing additional distillers corn oil from an ethanol production facility. The process broadly comprises obtaining a supply of whole stillage from the front-end of an ethanol production facility, after ethanol has been extracted therefrom: and introducing at least one diluent into the whole stillage, wherein the diluent has a lower percentage of solid than the whole stillage, and the mixture of the whole stillage and the diluent produces a distillers corn oil containing diluted stillage product for subsequent distillers corn oil production processes thereafter.

Another embodiment of the invention is a process for producing additional distillers corn oil from an ethanol production facility. This process broadly comprises: obtaining a supply of whole stillage from the front-end of an ethanol production facility, after ethanol has been extracted therefrom: introducing at least one diluent into the whole stillage, wherein the diluent has a lower percentage of solid than the whole stillage, and the mixture of the whole stillage and at least one diluent produces a distillers corn oil containing diluted stillage product: and introducing the distillers corn oil containing diluted stillage product to a first shearing flow separator, wherein the first shearing flow separator produces a first overflow stillage product and a first underflow stillage product for subsequent distillers corn oil production processes thereafter, wherein the first overflow stillage product has a higher percentage of distillers corn oil and a lower percentage of solid than the first underflow stillage product.

Another embodiment of the invention is a process for producing additional distillers corn oil and a high protein product from an ethanol production facility. This process broadly comprises: obtaining a supply of whole stillage from the front-end of an ethanol production facility after ethanol has been extracted therefrom: introducing at least one diluent into the whole stillage, wherein the diluent has a lower percentage of solid than the whole stillage, and the mixture of the whole stillage and at least one diluent produces a distillers corn oil containing diluted stillage product: introducing the distillers corn oil containing diluted stillage product to a first shearing flow separator, wherein the first shearing flow separator produces a first overflow stillage product and a first underflow stillage product, wherein the first overflow stillage product has a higher percentage of distillers corn oil and a lower percentage of solid than the first underflow stillage product: introducing the first underflow stillage product to a solid centrifuge separator, wherein the solid separator produces a thin stillage and a wet distiller grain, or wet cake: and introducing a first part of the first overflow stillage product to a first distillers corn oil centrifuge separator, wherein the first distillers corn oil centrifuge separator produces a first additional distillers corn oil product, a water phase product and a fine particle product with high protein of >40%.

Another embodiment of the invention is also a process for producing additional distillers corn oil and a high protein product from an ethanol production facility. This process broadly comprises obtaining a supply of whole stillage from the front-end of an ethanol production facility after ethanol has been extracted therefrom: introducing at least one diluent into the whole stillage, wherein the diluent has a lower percentage of solid than the whole stillage, and the mixture of the whole stillage and at least one diluent produces a distillers corn oil containing diluted stillage product: introducing the distillers corn oil containing diluted stillage product to a first shearing flow separator, wherein the first shearing flow separator produces a first overflow stillage product and a first underflow stillage product, wherein the first overflow stillage product has a higher percentage of distillers corn oil and a lower percentage of solid than the first underflow stillage product: introducing the first overflow stillage product to a second shearing flow separator, wherein in the second shearing flow separator produces a second overflow stillage product and a second underflow stillage product, wherein the second overflow stillage product has a higher percentage of distillers corn oil and a lower percentage of solid than the second underflow stillage product; introducing the first underflow stillage product and the second underflow stillage product to a solid centrifuge separator, wherein the solid separator produces a thin stillage and a wet distiller grain, or wet cake: and introducing a first part of the second overflow stillage product to a first distillers corn oil centrifuge separator, wherein the first distillers corn oil centrifuge separator produces a first additional distillers corn oil product, a water phase product, and a fine particle product with high protein of >40%.

Another embodiment of the invention is a process for producing additional distillers corn oil and a high protein product from an ethanol facility constructed in accordance with U.S. Pat. No. 8,633,003, also known as KFT. This process broadly comprises obtaining a supply of whole stillage from an ethanol production facility after ethanol has been extracted therefrom: pre-treating the whole stillage to convert hemicellulose portions of the whole stillage into sugars, the pre-treating including adding acid to the whole stillage to decrease its pH level, heating and pressurizing the whole stillage, holding the whole stillage under pressure and heat for a dwell time of 1-20 minutes, removing pressure from the whole stillage to cause flashing, and cooling the portion of whole stillage: adding enzymes to the whole stillage to convert cellulose portion of the whole stillage to sugars: fermenting the whole stillage utilizing yeast Saccharomyces cerevisiae to create a beer mixture: distilling the beer mixture to separate cellulosic ethanol to create a KFT whole stillage: introducing at least one diluent into the KFT whole stillage, wherein the diluent has a lower percentage of solid than the KFT whole stillage, and the mixture of the KFT whole stillage and at least one diluent produces a distillers corn oil containing diluted stillage product: introducing the distillers corn oil containing diluted stillage product to a first shearing flow separator, wherein the first shearing flow separator produces a first overflow stillage product and a first underflow stillage product: wherein the first overflow stillage product has a higher percentage of distillers corn oil and a lower percentage of solid than the first underflow stillage product: introducing the first overflow stillage product to a second shearing flow separator, wherein the second shearing flow separator produces a second overflow stillage product and a second underflow stillage product, wherein the second overflow stillage product has a higher percentage of distillers corn oil and a lower percentage of solid than the second underflow stillage product: introducing the first underflow stillage product and the second underflow stillage product to a solid centrifuge separator, wherein the solid separator produces a thin stillage and a wet distiller grain, or wet cake: and introducing a first part of the second overflow stillage product to a first distillers corn oil centrifuge separator, wherein the first distillers corn oil centrifuge separator produces a first additional distillers corn oil product, a water phase product and a fine particle product with high protein of >40%.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a schematic diagram of a conventional ethanol production facility.

FIG. 2 is a flow diagram depicting a process of producing additional ethanol from byproducts of the ethanol production facility of FIG. 1 with the teachings of U.S. Pat. No. 8,633,003, known as Kernal Fiber Technology, or KFT.

FIG. 3 is a schematic diagram of a system that may be used to implement the process of FIG. 2.

FIG. 4 is a schematic diagram depicting parts of the system of FIG. 3 in more detail.

FIG. 5 is a schematic diagram of the conventional ethanol production facility of FIG. 1 but focusing on DCO being produced.

FIG. 6 shows the plant data of different levels of DCO in two different wet cakes, one for a conventional ethanol plant and the other from a conventional plant following the teachings of U.S. Pat. No. 8,633,003.

FIG. 7 is a photo of a whole stillage sample after sitting on the table for a long time.

FIG. 8 shows an embodiment of the current invention for a conventional ethanol production facility.

FIG. 9 is a photo of two samples, an overflow sample on the left and an underflow sample on the right, produced by a shearing flow separator.

FIG. 10 shows particle size distributions of 2 samples from a shearing flow separator. The black line is the overflow sample, which has mostly fine particles of less than 10 microns. The blue line is the underflow sample, which has a broad particle size distribution and a lot of larger particles greater than 100 microns.

FIG. 11 shows another embodiment of the current invention for a conventional ethanol production facility.

FIG. 12 shows another embodiment of the current invention for a conventional ethanol production facility.

FIG. 13 shows another embodiment of the current invention for a conventional ethanol production facility with the teachings of U.S. Pat. No. 8,633,003.

The drawing figures do not limit the present invention to the specific embodiments disclosed and described herein. The drawings are not necessarily to scale, emphasis instead being placed upon clearly illustrating the principles of the current invention.

### DETAILED DESCRIPTION

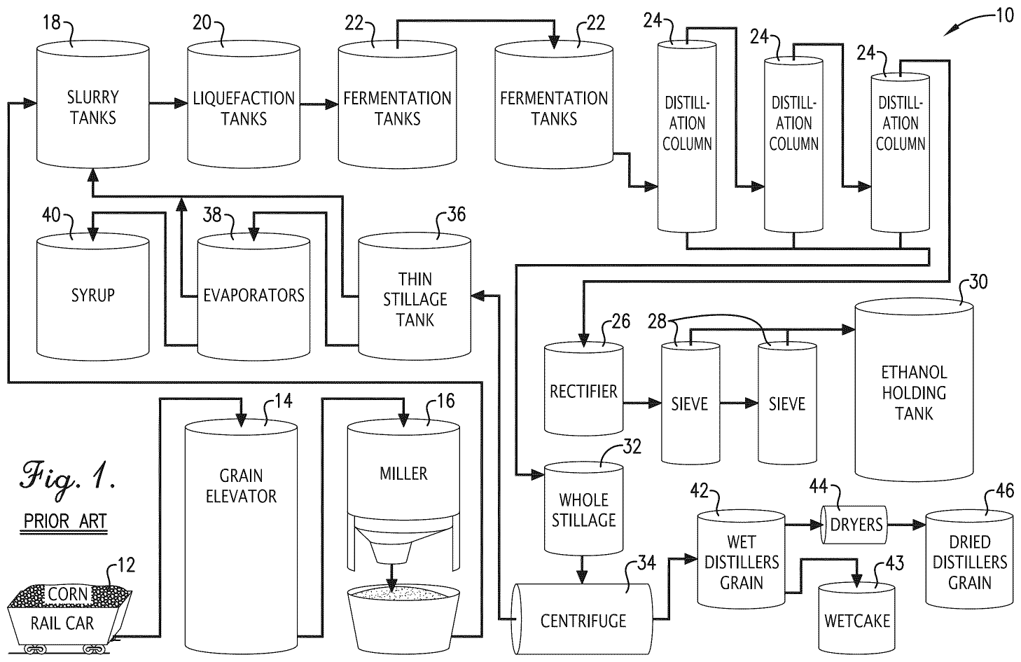

FIG. 1 is a schematic illustration of the primary components of a conventional ethanol production facility 10. The facility 10 is shown for purposes of describing aspects of the present invention and can be replaced, in whole or part, by other ethanol production facilities or components without departing from the scope of the present invention. Other ethanol production facilities and processes are described and illustrated in U.S. Pat. Nos. 6,660,506 and 7,527,941 and U.S. Patent Application No. 2004/0023349, all of which are incorporated in their entireties into the present application by reference.

Returning to FIG. 1, grain 12, such as corn, is first delivered to the ethanol production facility 10 by railcars, trucks, or other means. The grain may also be barley, rye, wheat, oats, sorghum, milo, canola, or soybeans. A sufficient supply of the grain to operate the production facility may be stored in one or more grain elevators 14, bins, or other storage vessels.

Ethanol production begins by milling or otherwise processing the grain into a fine powder or flour by a hammer mill or other milling machine 16. The milled grain is then mixed with water and enzymes in one or more slurry tanks 18 and held in these tanks or liquefaction tanks 20 for a sufficient amount of time to enable the enzymes to begin to breakdown the starch in the mixture into fermentable sugars.

The mixture is then passed to one or more fermentation tanks 22 where yeast is added. The fermenting process creates a mixture that contains alcohol, solids, and liquids and that is commonly referred to as “beer”. FIG. 1 only shows the main process steps following the flow of the solid/liquid steam: however, the fermentation process also produces a CO2 containing gas, which is not shown in FIG. 1.

The beer is then transferred to one or more distillation columns 24, also often referred to as “beer strippers”, which separate the alcohol from the solids and the liquids. The alcohol exits the top of these columns 24 and is transferred to one or more rectifiers 26 to remove moisture from the alcohol. The alcohol may also be passed to one or more molecular sieves 28 to remove even more moisture. The final alcohol is then transferred to one or more ethanol holding tanks 30 where it may be denatured before use as a fuel or fuel additive.

The liquid and solid mixture that remains in the distillation columns 24 after the alcohol has been removed is commonly referred to as “whole stillage” or simply “stillage”. The whole stillage falls to the bottom of the distillation columns 24 and is then transferred to one or more whole stillage holding tanks 32. The whole stillage may then be passed through one or more centrifuges 34 which separate it into a stream of “thin stillage” and a stream of “wet distillers grain”, WDG, or wet cake. The thin stillage is mostly liquid but may also contain a small amount of solid materials and distillers corn oil. A portion of thin stillage may be held in one or more tanks and is typically returned to the slurry tanks 18 or some other part of the front-end ethanol production facility 10 that requires water. This portion of the thin stillage recycle is called “backset”. The remainder of the thin stillage may also be transferred to one or more evaporators 38 to produce evaporated thin stillage, which is commonly referred to as “syrup”. The syrup may be held in one or more tanks 40 and be used as an animal feed additive. A distillers coil oil centrifuge can be used to separate distillers corn oil (DCO) from thin stillage or syrup as a separated product.

The wet distillers grain, which is often referred to as “wet cake”, may be held in storage facilities 42, 43 and sold as a livestock feed. Some of the wet distillers grain may also be transferred to one or more dryers 44 to remove liquid therefrom to produce dried distillers grain, which may also be stored in one or more tanks 46 and used as livestock feed. In addition, some of the syrup can be dried with the wet distillers grains to produce distiller's dried grains with soluble (DDGS).

The above described ethanol production facility 10 does not attempt to produce additional ethanol from the whole stillage removed from the distillation columns 24. Instead, the whole stillage is just a byproduct of the ethanol production process and is either used as livestock feed, make-up water, and/or is discarded. The processes of the present invention produce additional useable ethanol from the fiber portions of the whole stillage and any leftover starch after the main ethanol extraction is complete. The processes of the present invention also increase the protein content of the final byproducts, thus improving their nutrient value when used as livestock feed.

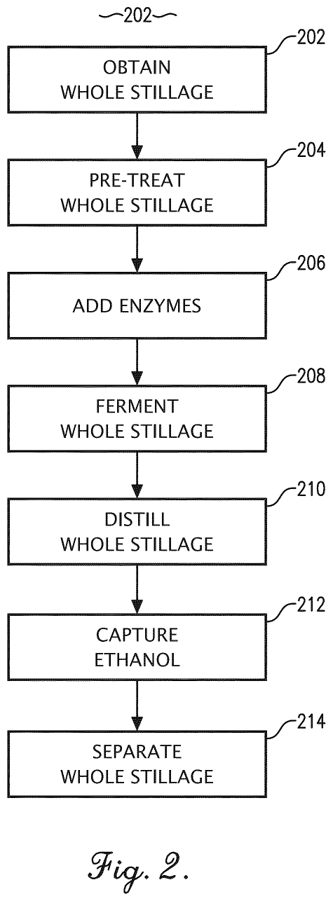

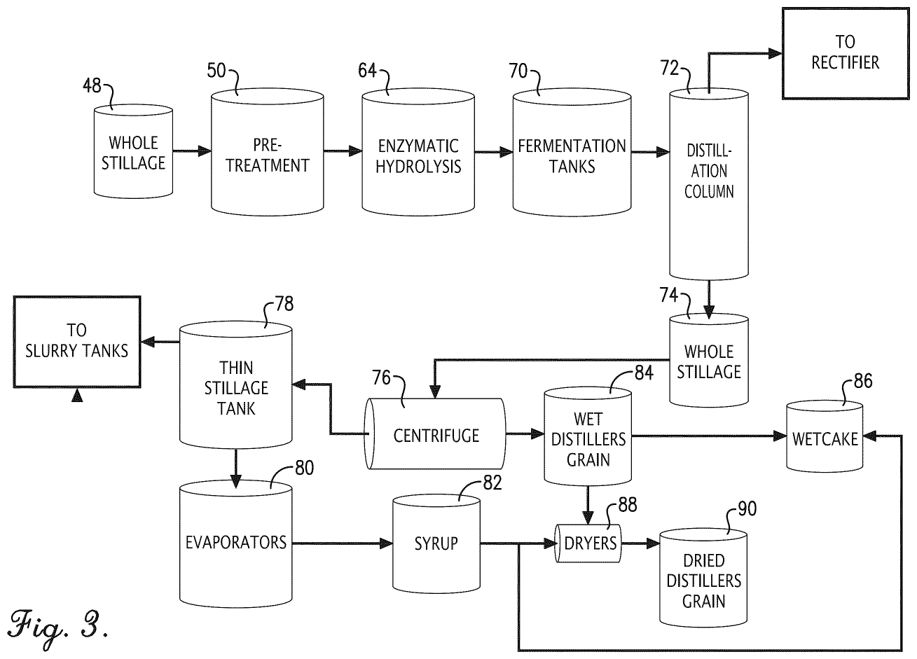

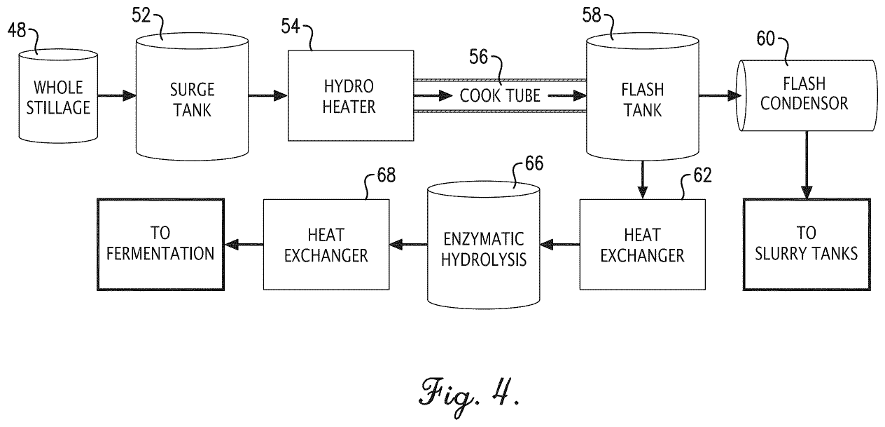

The flow chart of FIG. 2 shows the steps in a process 200 for producing additional ethanol from whole stillage as disclosed in U.S. Pat. No. 8,633,003, commonly known as Kernel Fiber Technology, or KFT. The schematic diagrams of FIGS. 3 and 4 depict equipment that may be used to implement the process 200 and other embodiments of the process. The illustrated equipment may be replaced with other equipment without departing from the scope of the invention.

The process 200 starts by obtaining whole stillage as depicted in block 202. The whole stillage may be obtained from the distillation columns 24 or the whole stillage tanks 32 shown in FIG. 1 or elsewhere in the ethanol production facility 10 and may be held in one or more tanks 48 as depicted in FIG. 3. At this point in the process, the whole stillage has already been subjected to: 1) a long soak time in the liquefaction tanks 20 and fermenting tanks 22, 2) heating in the distillation steps, and 3) chemical reactions from the chemicals added throughout the ethanol production process. These steps help to facilitate the breakdown of the fiber in the whole stillage. The whole stillage is further treated in the process 200 of the present invention to continue the breakdown of the fiber for extracting additional ethanol.

The whole stillage is then subjected to a pre-treatment process as depicted in block 204 of FIG. 2. In one embodiment, the pre-treatment process may be implemented with the equipment 50 generally illustrated in FIG. 3 and shown in more detail in FIG. 4. Referring to FIG. 4, the whole stillage is first transferred to a surge tank 52 or other vessel. Sulfuric acid or another acid is then added to the surge tank 52 to decrease the pH level of the whole stillage to promote dilute acid hydrolysis. Sufficient acid is supplied to decrease the pH of the whole stillage to 1.0 to 4.5 and preferably to 1.8.

The pre-treatment portion of the process 200 continues by heating and pressurizing the whole stillage mixture. In one embodiment, the heating is performed in a hydro-heater 54 shown in FIG. 4 where high pressure steam is injected into the mixture to increase its temperature to 215° F. to 260° F., with the best results at 260º F. Higher temperatures up to 300° F. may be even more beneficial, but temperatures above 260º F. may be difficult to achieve economically. Heating by steam injection is beneficial because it results in cavitation of the mixture which further disrupts the structure of the fiber in the whole stillage which aids in subsequent processing of the whole stillage.

During the heating process, the whole stillage is also pressurized in the hydro-heater 54, a cook tube 56, or other vessel to a pressure in excess of the vapor pressure to prevent the mixture from boiling. The heated mixture is then held at the elevated temperature and pressure for 2-20 minutes. Applicant has discovered that retention times beyond 20 minutes don't provide substantial additional benefits.

The pre-treatment portion of the process 200 continues by transferring the whole stillage mixture to a flash tank 58 where its pressure is rapidly dropped, causing the mixture to boil and flash off steam. This rapid boiling causes further rupturing of the fiber structure of the whole stillage to further expose the cellulose and hemicellulose of the whole stillage. The steam from the flash tank 58 may be captured in a flash condensor 60 and used as make-up water in the slurry tanks or elsewhere in the ethanol production facility 10 as depicted in FIG. 4.

Afterward, the whole stillage is cooled in one or more heat exchanges 62 to a temperature appropriate for the subsequent enzymatic hydrolysis process. Pentose sugars, such as xylose, may be produced from the hemicellulose by this portion of the method.

The mixture is then subjected to an enzymatic hydrolysis process as depicted in block 212 of FIG. 2. In one embodiment, the enzymatic hydrolysis process may be implemented with the equipment 64 shown generally in FIG. 3 and shown in more detail in FIG. 4. The enzymatic hydrolysis primarily converts the cellulose portions of the fiber to usable sugars but also converts some of the hemicellulose to sugars. The whole stillage is first transferred to one or more tanks 66 shown in FIG. 4 where enzymes are added. Ammonia may also be added to increase the pH to a level conducive to the activity of the enzymes. In one embodiment, the whole stillage is held at a temp of 150° F. -160° F., with a preferred temp of 158° F. and a pH level of approximately 4.5. The whole stillage is then cooled in one or more heat exchangers 68 and, if necessary, pH corrected to a level of 4.8 to avoid denaturing the enzymes. Hexose sugars, such as glucose, may be produced from the cellulose by the enzymatic hydrolysis.

The whole stillage is next fermented as depicted by block 208 in FIG. 2 and equipment 70 in FIG. 3. The fermentation of the mixed sugars produced in the pre-treatment and enzymatic hydrolysis steps described above requires an organism different than the yeast used in the fermentation step of the main ethanol production facility 10. Saccharomyces cerevisiae is only able to ferment hexose sugars, and therefore cannot use the pentose sugars unlocked from the hemicelluloses. Another yeast or bacteria is needed to perform this step.

After fermentation, whole stillage is distilled and separated as depicted by block 210 in FIG. 2 to remove ethanol from the whole stillage. The distillation may be performed in one or more distillation columns 72 depicted in FIG. 3. The distillation portion of the process 200 is similar to the distillation that occurs in the distillation columns 24 of the main ethanol production facility 10. The ethanol or alcohol exits the top of the columns 72 and is transferred to one or more rectifiers to remove moisture from the alcohol. The alcohol may also be passed to one or more molecular sieves to remove even more moisture. The final alcohol is then transferred to one or more ethanol holding tanks where it may be denatured before use as a fuel or fuel additive. The alcohol from the distillation columns 72 may be transferred to the rectifiers 26 and sieves 28 of the main ethanol production facility 10 where it is co-mingled with the ethanol from the distillation columns 24 or it may be purified by its own dedicated rectifiers and sieves.

The whole stillage that remains in the distillation columns 72 after the alcohol has been removed falls to the bottom of the distillation columns and is then transferred to one or more whole stillage holding tanks 74. The whole stillage at this point is similar to the whole stillage obtained at the beginning of the process 200 except that it has less solids and higher protein. The whole stillage may then be passed through one or more centrifuges 76 which separate the whole stillage into a stream of thin stillage and a stream of wet distillers grain. The thin stillage may be held in one or more tanks 78 and a portion is typically returned to the slurry tanks 18 or some other part of the ethanol production facility 10 that requires water. This portion of thin stillage recycle is called “backset”. The remaining of the thin stillage may also be transferred to one or more evaporators 80 to produce evaporated thin stillage, which is commonly referred to as “syrup”. The syrup may be held in one or more tanks 82 and be used as an animal feed additive.

The wet distillers grain, which is often referred to as “wetcake”, may be held in storage facilities 84, 86 and also sold as a livestock feed. Some of the wet distillers grain may be passed through one or more dryers 88 to remove liquid therefrom to produce dried distillers grain, which may be stored in one or more tanks 90 and used as dry livestock feed. The syrup from the tanks 82 may also be dehydrated in the dryers 88 forming dried distillers grain with soluble (DDGS).

A conventional corn to ethanol process produces the following from one bushel of corn:

| Product | Yield | |
|---|---|---|
| Ethanol | 2.75 | Gallons per Bushel |
| DDGS | 16.4 | Pounds per Bushel |
| Corn Oil | 0.75 | Pounds per Bushel |

In contrast, an embodiment of the process 200 of the present invention produces the following from one bushel of corn:

| Product | Yield | |
|---|---|---|
| Ethanol | 3.02 | Gallons per Bushel |
| DDGS | 11.3 | Pounds per Bushel |
| Corn Oil | 1.05 | Pounds per Bushel |

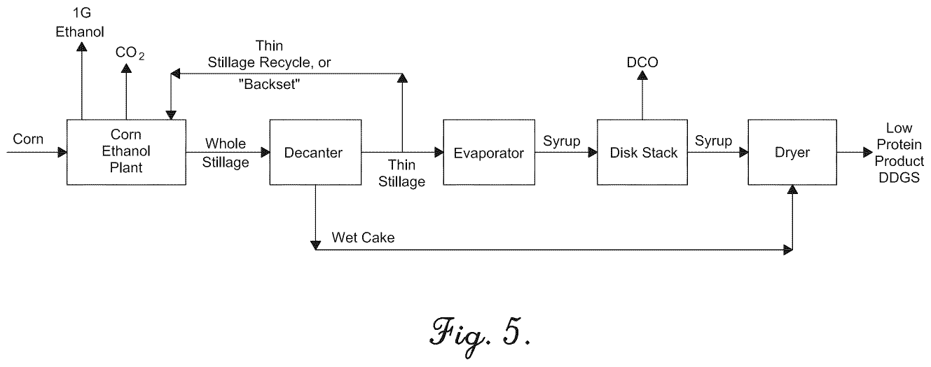

FIG. 5 shows a flow diagram similar to one in FIG. 1 but focuses on the back-end DCO production processes. It shows that that most ethanol plants start the back-end DCO separation process with a centrifuge solid separation device, such as a decanter, for producing wet distillers grain, or wet cake, and thin stillage, followed by producing syrup from evaporator and producing DCO from syrup. The shortcoming with these processes is that a significant DCO in the whole stillage is “lost” to the wet distillers grain in the first step, which became no longer available for the final DCO production from syrup. Furthermore, FIG. 5 shows the recycling of thin stillage, or backset, to the front end of the ethanol plant, which contains DCO. This further worsens the DCO loss to the wet cake in the first step of the DCO production process.

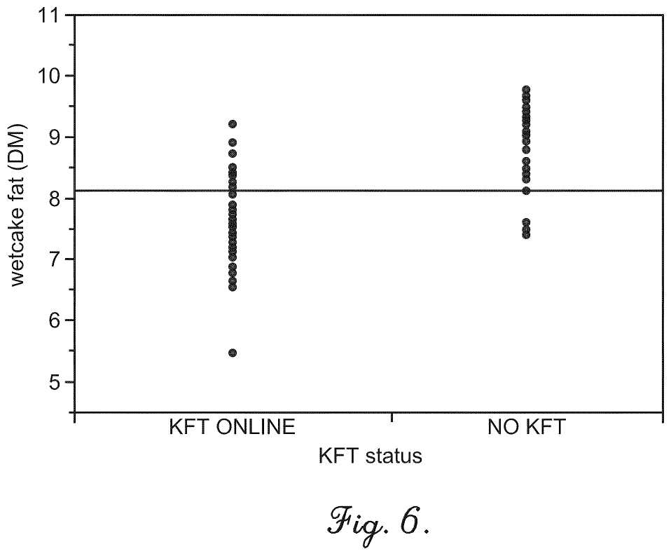

FIG. 6 shows different levels of DCO from two different wet cakes from two different corn ethanol plants. There is about 9% DCO in the conventional ethanol plant's wet cake whereas there is about 7% DCO in the wet cake from a conventional ethanol plant with KFT technology. KFT DCO yield about 1.05 lb/bushel, which is higher than the Q3 2020 industry median of 0.79 lb/bushel. This confirms that reducing DCO loss to the wet cake in the overall DCO production process is the key to increase overall DCO production. The current invention includes effective processes and methods, much superior to KFT, to minimize the DCO loss to the wet cake via reducing the amount of DCO sent to the decanter for making the wet cake, thus increasing the amount of DCO available in process stream(s) for the DCO production.

The whole stillage is a high viscosity, three-phase mixture, having water as the continuous phase, with suspended solid particles and buoyant DCO droplets. The viscosity of the whole stillage is very high, similar to yogurt, which makes it difficult to directly process the whole stillage for producing DCO. Some corn ethanol plants have used a three-phase centrifuge tri-canter, instead of the two-phase solid centrifuge of a decanter, to directly process the whole stillage for producing DCO, but with limited success.

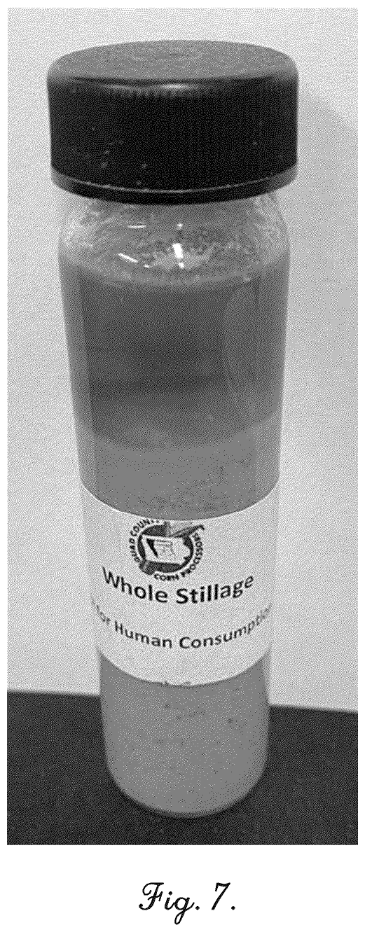

FIG. 7 shows a photo of a whole stillage sample after settling on the table for many months. This photo confirms that, even though the whole stillage has very high viscosity, it is possible to separate DCO from the whole stillage: however, relying solely on gravitational settling takes too long to be practical or economical. The current invention employs a unique approach of using at least one diluent, which has a lower suspended solid percentage than the whole stillage, to form a mixture of the whole stillage and diluent. This produces a distillers corn oil containing diluted stillage product, which has a much lower viscosity and, more importantly, makes DCO separation more effective and economical. In addition, the current invention discovered that stillage is a shear-thinning material and, by using processes and methods which induce a shearing flow, the viscosity of the stillage is further reduced, making DCO separation even more effective and economical. The combination of these discoveries and solutions enables the current invention to minimize the DCO loss to the wet cake via reducing the amount of DCO in the process streams sent to the decanter for making the wet cake, thus increasing the amount of DCO available in process stream(s) for the DCO production.

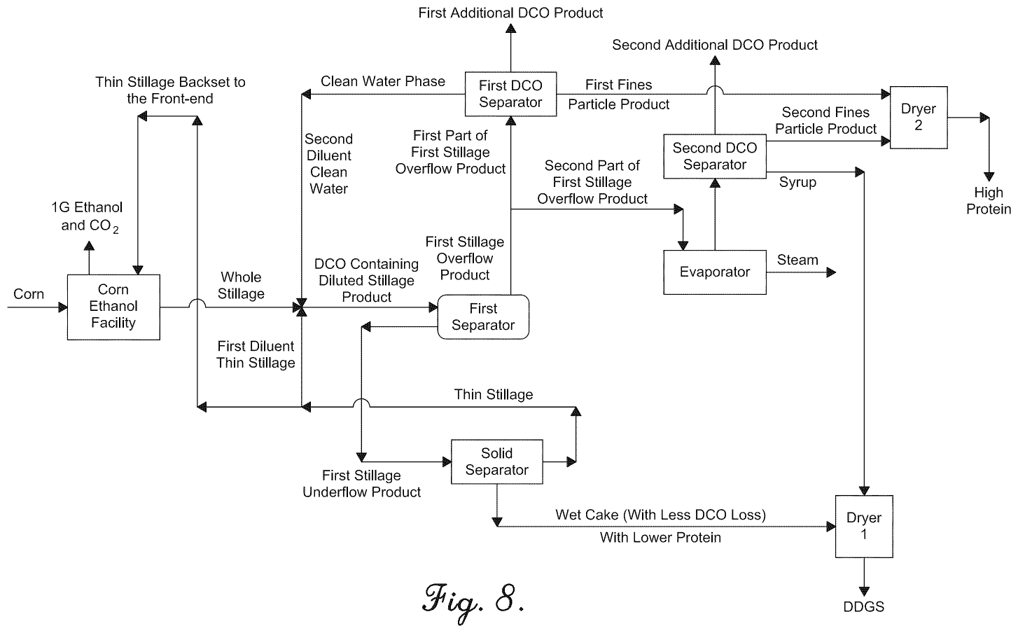

FIG. 8 is a schematic flow diagram for an embodiment of the current invention for producing more DCO and a high protein corn product from a conventional corn ethanol production facility. The corn ethanol production facility receives corn feed and produces conventional ethanol, CO2, and whole stillage having a solid percentage between 10 and 20 weight percent. The invention then introduces at least one diluent into the whole stillage, wherein the diluent has a lower percentage of solid than the whole stillage. As explained herein, the diluent may be thin stillage from a decanter, clean water phase from a DCO separator, or another substance that has a lower percentage of solid than the whole stillage. The mixture of the whole stillage and at least one diluent produces a distillers corn oil (DCO) containing diluted stillage product. The DCO containing diluted stillage product is then introduced to a first shearing flow separator, wherein the first shearing flow separator produces a first overflow stillage product and a first underflow stillage product, wherein the first overflow stillage product has a higher DCO percentage and a lower percentage of solid than the first underflow stillage product. The invention then introduces the first underflow stillage product to a solid centrifuge separator, wherein the solid separator produces a thin stillage and a wet distiller grain, or wet cake. A first part of the first overflow stillage product is then introduced to a first distillers corn oil centrifuge separator, wherein the first distillers corn oil centrifuge separator produces a first additional DCO product, a water phase product, and a fine particle product with high protein of >40%. A second part of the first overflow stillage product is then introduced to an evaporation device, wherein the evaporation device produces steam and a DCO containing syrup. The DCO containing syrup is then introduced to a second distillers corn oil centrifuge separator, wherein the second distillers corn oil centrifuge separator produce a syrup and a second additional DCO product.

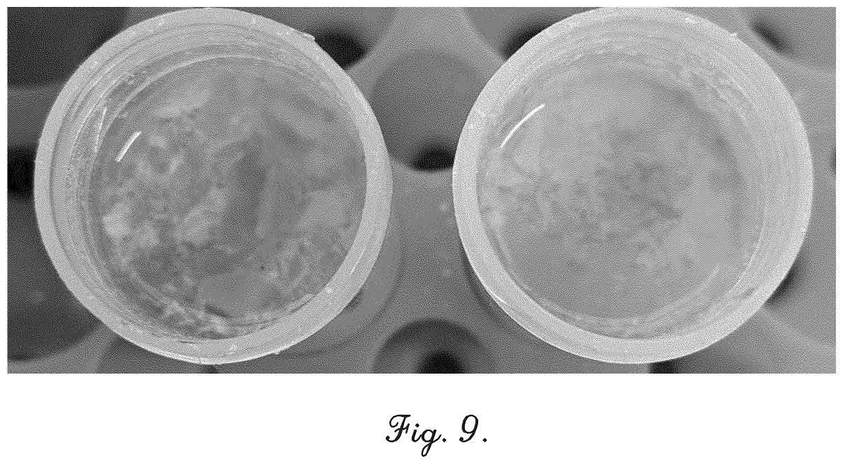

FIG. 9 shows a photo of two samples produced from the first shearing flow separator, the overflow sample on the left and the underflow sample on the right. The photo shows that the overflow sample has more orange color DCO floating on the top than the underflow sample. This confirms that the current invention can minimize the DCO loss to the wet cake via reducing the amount of DCO in the underflow process stream going to the decanter for making the wet cake and increasing the amount of DCO available in the overflow process stream going to the disk stacks for the DCO production.

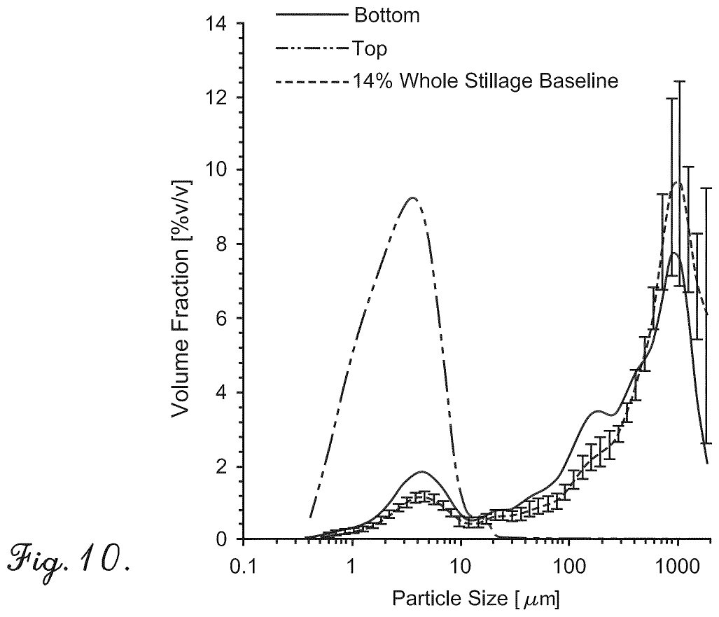

FIG. 10 shows particle size distributions of 2 samples from the first shearing flow separator. The blue line is the underflow sample, which has a broad particle size distribution and lots of larger particles greater than 100 microns. The black line is the overflow sample, which has mostly fine particles of less than 10 microns. This not only confirms the capability of the first shearing flow separator to produce an underflow with a higher solid percentage than the overflow, but also confirms a hydro cyclone separator's capability of isolating fine particles to the overflow. Separate measurements of the protein contents of the two samples confirm that the fine particle product has high protein of >40%, whereas the underflow with lots of large particles has low protein.

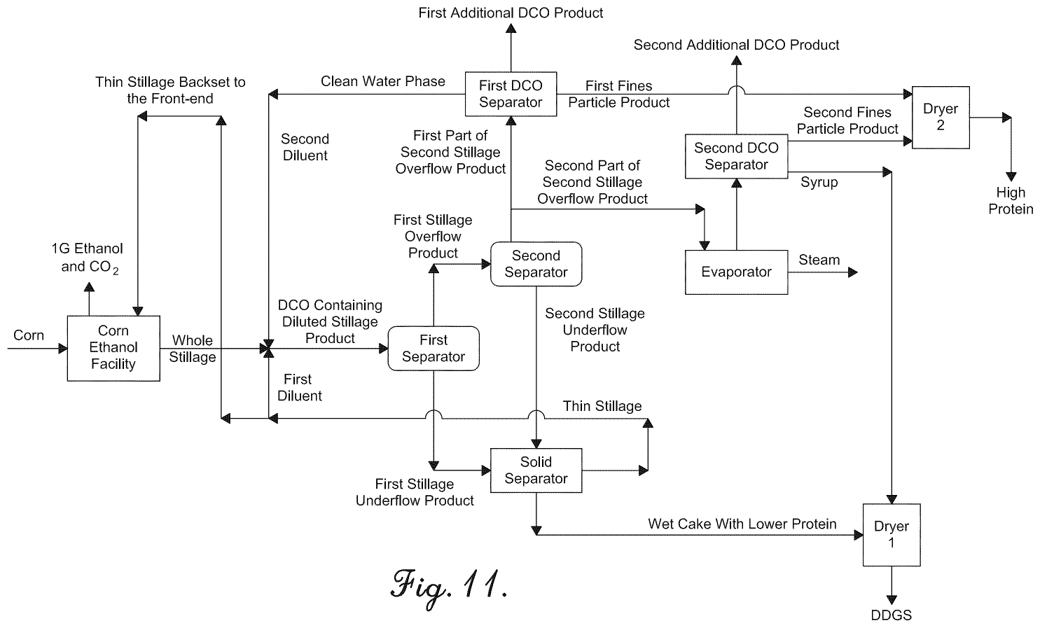

FIG. 11 is a schematic flow diagram for an embodiment of the current invention for producing more DCO and a high protein corn product from a conventional corn ethanol production facility. The corn ethanol production facility takes corn feed and produces conventional ethanol, CO2, and whole stillage having a solid percentage between 10 and 20 weight percent. At least one diluent is then introduced into the whole stillage, wherein the diluent has a lower percentage of solid than the whole stillage, and the mixture of the whole stillage and at least one diluent produces a distillers corn oil (DCO) containing diluted stillage product. The DCO containing diluted stillage product is then introduced to a first shearing flow separator, wherein the first shearing flow separator produces a first overflow stillage product and a first underflow stillage product, wherein the first overflow stillage product has a higher DCO percentage and a lower percentage of solid than the first underflow stillage product. The first overflow stillage product is then introduced to a second shearing flow separator, wherein the second shearing flow separator produces a second overflow stillage product and a second underflow stillage product, wherein the second overflow stillage product has a higher percentage of distillers corn oil and a lower percentage of solid than the second underflow stillage product. The first underflow stillage product and the second underflow stillage product are introduced to a solid centrifuge separator, wherein the solid separator produces a thin stillage and a wet distiller grain, or wet cake. A first part of the second overflow stillage product is introduced to a first distillers corn oil centrifuge separator, wherein the first distillers corn oil centrifuge separator produces a first additional DCO product, a water phase product, and a fine particle product with high protein of >40%. A second part of the second overflow stillage product is then introduced to an evaporation device, wherein the evaporation device produces steam and a DCO containing syrup. The DCO containing syrup is then introduced to a second distillers corn oil centrifuge separator, wherein the second distillers corn oil centrifuge separator produce a syrup and a second additional DCO product.

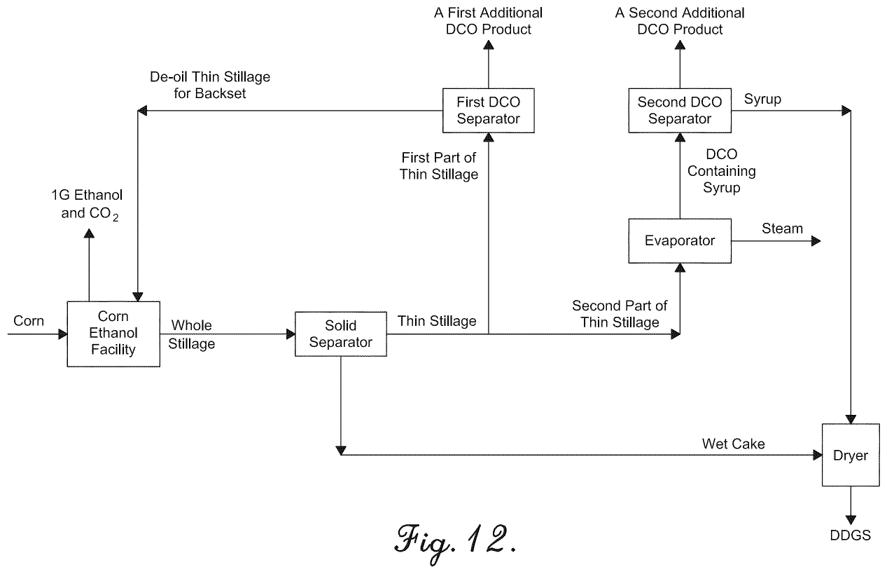

FIG. 12 is another schematic flow diagram for an embodiment of the current invention for producing more DCO from a conventional corn ethanol production facility. A method of the invention obtains a supply of whole stillage from the front-end of an ethanol production facility after ethanol has been extracted therefrom. The whole stillage is then introduced to a solid centrifuge separator, wherein the solid separator produces a wet distiller grain, or wet cake, and a thin stillage. A first part of the thin stillage is introduced to a first distillers corn oil centrifuge separator, wherein the distiller corn oil separator produces a first additional distiller corn oil product and a de-oil thin stillage. The de-oil thin stillage is introduced as backset to the front-end of the ethanol production facility. A second part of the thin stillage is introduced to an evaporator, wherein the evaporator produces steam and a distillers corn oil containing syrup. The distillers corn oil containing syrup is introduced to a second distillers corn oil centrifuge separator, wherein the second distillers corn oil centrifuge separator produces a syrup and a second additional distillers corn oil.

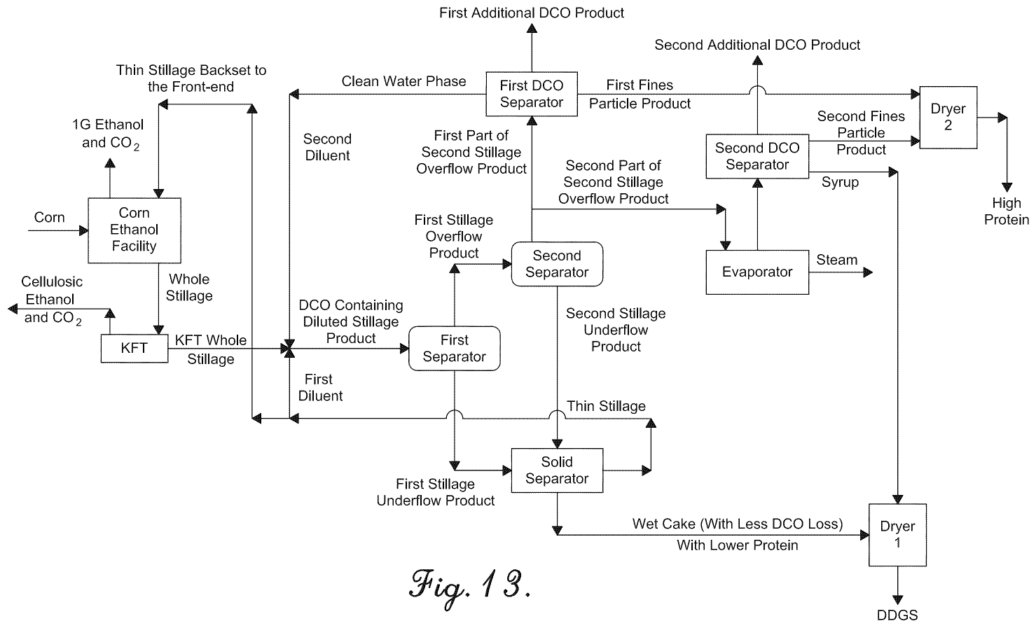

FIG. 13 is another schematic flow diagram for an embodiment of the current invention for producing more DCO and a high protein corn product from an ethanol facility constructed in accordance with U.S. Pat. No. 8,633,003, also known as KFT. A method obtains a supply of whole stillage from the front-end of an ethanol production facility after ethanol has been extracted therefrom. The whole stillage is pre-treated to convert hemicellulose portions of the whole stillage into sugars, the pre-treating including:
i. adding acid to the whole stillage to decrease its pH level;
ii. heating and pressurizing the whole stillage;
iii. holding the whole stillage under pressure and heat for a dwell time of 1-20 minutes;
iv. removing pressure from the whole stillage to cause flashing: and
v. cooling the portion of whole stillage.
   Enzymes are then added to the whole stillage to convert cellulose portion of the whole stillage to sugars. The whole stillage is fermented utilizing yeast Saccharomyces cerevisiae to create a beer mixture. The beer mixture is distilled to separate cellulosic ethanol for producing a Kernal Fiber Technology (KFT) whole stillage.

The method further comprises at least one diluent into the KFT whole stillage, wherein the diluent has a lower percentage of solid than the KFT whole stillage, and the mixture of the KFT whole stillage and at least one diluent produces a distillers corn oil containing diluted stillage product. The distillers corn oil containing diluted stillage product is then introduced to a first shearing flow separator, wherein the first shearing flow separator produces a first overflow stillage product and a first underflow stillage product. The first overflow stillage product has a higher percentage of distillers corn oil and a lower percentage of solid than the first underflow stillage product. The first overflow stillage product is introduced to a second shearing flow separator, wherein the second shearing flow separator produces a second overflow stillage product and a second underflow stillage product. The second overflow stillage product has a higher percentage of distillers corn oil and a lower percentage of solid than the second underflow stillage product. The first underflow stillage product and the second underflow stillage product are introduced to a solid centrifuge separator, wherein the solid separator produces a thin stillage and a wet distiller grain, or wet cake. A first part of the second overflow stillage product is introduced to a first distillers corn oil centrifuge separator, wherein the first distillers corn oil centrifuge separator produces a first additional distillers corn oil product, a water phase product, and a fine particle product with high protein. A second part of the second overflow stillage product is introduced to an evaporation device, wherein the evaporation device produces steam and a DCO containing syrup. The DCO containing syrup is introduced to a second distillers corn oil centrifuge separator, wherein the second distillers corn oil centrifuge separator produce a syrup and a second additional DCO product.

### ADDITIONAL CONSIDERATIONS

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A method of producing additional distillers corn oil and a high protein product from an ethanol production facility, the method comprising:
a) obtaining a supply of whole stillage from the ethanol production facility after ethanol has been extracted therefrom; and
b) introducing at least one diluent into the whole stillage, wherein the diluent has a lower percentage of solid than the whole stillage, and the mixture of the whole stillage and the diluent produces a distillers corn oil containing diluted stillage product.

2. The method as set forth in claim 1, wherein the whole stillage has a solid percentage between 10 and 20 weight percent.

3. The method as set forth in claim 1, the method further comprising:
introducing the distillers corn oil containing diluted stillage product to a first shearing flow separator, wherein the first shearing flow separator produces a first overflow stillage product and a first underflow stillage product for subsequent distillers corn oil production processes thereafter; wherein the first overflow stillage product has a higher percentage of distillers corn oil and a lower percentage of solid than the first underflow stillage product.

4. The method as set forth in claim 3, the method further comprising:
a) introducing the first underflow stillage product to a solid centrifuge separator, wherein the solid centrifuge separator produces a thin stillage and a wet distiller grain, or wet cake; and
b) introducing a first part of the first overflow stillage product to a first distillers corn oil centrifuge separator, wherein the first distillers corn oil centrifuge separator produces a first additional distillers corn oil product and a water phase product.

5. The method as set forth in claim 4, wherein the first distiller corn oil separator further produces a first fine particle product, wherein the first fine particle product has a high protein concentration of greater than 40%.

6. The method as set forth in claim 4, the method further comprising:
a) introducing a first part of the thin stillage as a first diluent into the whole stillage; and
b) introducing the water phase product as a second diluent into the whole stillage, wherein the mixture of the whole stillage, the first diluent and the second diluent produces the distillers corn oil containing diluted stillage product.

7. The method as set forth in claim 4, the method further comprising:
introducing a second part of the thin stillage as backset to a front-end of the ethanol production facility.

8. The method as set forth in claim 4, the method further comprising:
a) introducing a second part of the first overflow stillage product to an evaporation device, wherein the evaporation device produces steam and a distiller corn oil containing syrup, and
b) introducing the distiller corn oil containing syrup to a second distillers corn oil centrifuge separator, wherein the second distillers corn oil centrifuge separator produces a syrup and a second additional distillers corn oil product.

9. A method of producing additional distillers corn oil and a high protein product from an ethanol production facility, the method comprising:
a) obtaining a supply of whole stillage from the ethanol production facility after ethanol has been extracted therefrom;
b) introducing at least one diluent into the whole stillage, wherein the diluent has a lower percentage of solid than the whole stillage, and the mixture of the whole stillage and the diluent produces a distillers corn oil containing diluted stillage product;
c) introducing the distillers corn oil containing diluted stillage product to a first shearing flow separator, wherein the first shearing flow separator produces a first overflow stillage product and a first underflow stillage product: wherein the first overflow stillage product has a higher percentage of distillers corn oil and a lower percentage of solid than the first underflow stillage product;
d) introducing the first overflow stillage product to a second shearing flow separator, wherein the second shearing flow separator produces a second overflow stillage product and a second underflow stillage product, wherein the second overflow stillage product has a higher percentage of distillers corn oil and a lower percentage of solid than the second underflow stillage product;
e) introducing the first underflow stillage product and the second underflow stillage product to a solid centrifuge separator, wherein the solid centrifuge separator produces a thin stillage and a wet distiller grain, or wet cake; and
f) introducing a first part of the second overflow stillage product to a first distillers corn oil centrifuge separator, wherein the first distillers corn oil centrifuge separator produces a first additional distillers corn oil product and a water phase product.

10. The method as set forth in claim 9, the method further comprising:
a) introducing a first part of the thin stillage as a first diluent into the whole stillage; and
b) introducing the water phase product as a second diluent into the whole stillage, and the mixture of the whole stillage, the first diluent and the second diluent produces the distillers corn oil containing diluted stillage product for subsequent distillers corn oil production processes.

11. The method as set forth in claim 9, wherein the first distiller corn oil separator further produces a first fine particle product, wherein the first fine particle product has a high protein concentration of greater than 40%.

12. The method as set forth in claim 9, the method further comprising:
introducing a second part of the thin stillage as backset to a front-end of the ethanol production facility.

13. The method as set forth in claim 9, the method further comprising:
a) introducing a second part of the second overflow stillage product to an evaporation device, wherein the evaporation device produces steam and a distiller corn oil containing syrup; and
b) introducing the distiller corn oil containing syrup to a second distillers corn oil centrifuge separator, wherein the second distillers corn oil centrifuge separator produces a syrup and a second additional distillers corn oil product.

14. A method of producing additional distillers corn oil from an ethanol production facility, the method comprising:
a) obtaining a supply of whole stillage from the ethanol production facility after ethanol has been extracted therefrom;
b) introducing the whole stillage to a solid centrifuge separator, wherein the solid separator produces a wet distiller grain, or wet cake, and a thin stillage;
c) introducing a first part of thin stillage to a first distillers corn oil centrifuge separator, wherein the distiller corn oil separator produce a first additional distiller corn oil product and a de-oil thin stillage;
d) introducing the de-oil thin stillage as backset to a front-end of the ethanol production facility;
e) introducing a second part of the thin stillage to an evaporator, wherein the evaporator produces steam and a distillers corn oil containing syrup; and
f) introducing the distillers corn oil containing syrup to a second distillers corn oil centrifuge separator, wherein the second distillers corn oil centrifuge separator produces a syrup and a second additional distillers corn oil.

15. A method of producing additional distillers corn oil and a high protein product from an ethanol production facility, the method comprising:
a) obtaining a supply of whole stillage from the ethanol production facility after ethanol has been extracted therefrom;
b) pre-treating the whole stillage to convert hemicellulose portions of the whole stillage into sugars, the pre-treating including:
i. adding acid to the whole stillage to decrease its pH level,
ii. heating and pressurizing the whole stillage,
iii. holding the whole stillage under pressure and heat for a dwell time of 1-20 minutes,
iv. removing pressure from the whole stillage to cause flashing, and
v. cooling the portion of whole stillage;
c) adding enzymes to the whole stillage to convert cellulose portion of the whole stillage to sugars;
d) fermenting the whole stillage utilizing yeast Saccharomyces cerevisiae to create a beer mixture;
e) distilling the beer mixture to separate cellulosic ethanol for producing a Kernal Fiber Technology (KFT) whole stillage;
f) introducing at least one diluent into the KFT whole stillage, wherein the diluent has a lower percentage of solid than the KFT whole stillage, and the mixture of the KFT whole stillage and the diluent produces a distillers corn oil containing diluted stillage product;
g) introducing the distillers corn oil containing diluted stillage product to a first shearing flow separator, wherein the first shearing flow separator produces a first overflow stillage product and a first underflow stillage product, wherein the first overflow stillage product has a higher percentage of distillers corn oil and a lower percentage of solid than the first underflow stillage product;
h) introducing the first overflow stillage product to a second shearing flow separator, wherein in the second shearing flow separator produces a second overflow stillage product and a second underflow stillage product, wherein the second overflow stillage product has a higher percentage of distillers corn oil and a lower percentage of solid than the second underflow stillage product;
i) introducing the first underflow stillage product and the second underflow stillage product to a solid centrifuge separator, wherein the solid separator produces a thin stillage and a wet distiller grain, or wet cake; and
j) introducing a first part of the second overflow stillage product to a first distillers corn oil centrifuge separator, wherein the first distillers corn oil centrifuge separator produces a first additional distillers corn oil product and a water phase product.

16. The method as set forth in claim 15, the method further comprising:
a) introducing a first part of the thin stillage as a first diluent into the whole stillage; and
b) introducing the water phase product as a second diluent into the whole stillage, and the mixture of the whole stillage, the first diluent and the second diluent produces the distillers corn oil containing diluted stillage product for subsequent distillers corn oil production processes thereafter.

17. The method as set forth in claim 15, wherein the first distiller corn oil separator further produces a first fine particle product, wherein the first fine particle product has a high protein concentration of greater than 40%.

18. The method as set forth in claim 15, the method further comprising:
introducing a second part of the thin stillage as backset to a front-end of the ethanol production facility.

19. The method as set forth in claim 15, wherein the method further comprising:
a) introducing a second part of the second overflow stillage product to an evaporation device, wherein the evaporation device produces steam and a distiller corn oil containing syrup; and
b) introducing the distiller corn oil containing syrup to a second distillers corn oil centrifuge separator, wherein the second distillers corn oil centrifuge separator produce a syrup and a second additional distillers corn oil product.
